(19) United States
(12) Patent Application Publication
(10) Pub. No.: US 2011/0318816 A1
(43) Pub. Date: Dec. 29, 2011

(54) SYSTEM FOR MODERATING THE TEMPERATURE OF A MEDIUM FOR GROWING MICROALGAE

(76) Inventor: David A. Hazlebeck, El Cajon, CA (US)

(21) Appl. No.: 12/823,987

(22) Filed: Jun. 25, 2010

(51) Int. Cl. C12N 1/12; C12M 1/00

(52) U.S. Cl. 435/257.1; 435/303.1

(57) ABSTRACT

A system is provided for moderating the temperature of a medium for growing microalgae and for distilling fresh water. In the system, the medium flows through a conduit having two ends and a bottom. As the sunlight passes into the medium, the algae grows and the medium is heated. For the system, an impermeable first liner is extended across the conduit to cover the bottom of the conduit. Further, a gas permeable/liquid impermeable second liner is extended across the conduit to cover the bottom of the conduit. With the second liner positioned on top of the first liner, vapor medium is only able to pass through the second liner. Also, a condensing mechanism is positioned between the first and second liners. Any vapor medium that passes through the second liner is condensed into fresh water. As a result, fresh water is distilled and the medium is cooled through condensation.

### FIELD OF THE INVENTION

The present invention pertains generally to methods for growing algae. More particularly, the present invention pertains to the use of a system for moderating the temperature of a medium used in a conduit for growing algae, and for distilling fresh water. The present invention is particularly, but not exclusively, useful as a system for removing a vapor medium from the medium in the conduit and for distilling that vapor medium to obtain usable fresh water.

### BACKGROUND OF THE INVENTION

Because algae is known to be one of the most efficient plants for converting solar energy into cell growth, it is of particular interest as a biofuel source. Importantly, the use of algae as a biofuel source presents no particularly exceptional problems, i.e., biofuel can be processed from oil in algae as easily as from oils in land-based plants. As the demand for biofuels increases, the need for more cost effective and efficient systems correspondingly increases.

While algae can efficiently transform solar energy into chemical energy via a high rate of cell growth, it has been difficult to create environments in which algae cell growth rates are optimized. Specifically, the conditions necessary to facilitate a fast growth rate for algae cells in large-scale operations have been found to be expensive to create. While sunlight can be cheaply and easily fed to algae, it can present other problems in processing. For example, only about 3% of the sunlight that reaches a medium holding microalgae is used by the microalgae chemically. Consequently, the rest of the solar energy (i.e. 97%) is absorbed by the medium as heat. Taking into consideration the large-scale conduits that algae growing systems use, this absorption of heat can lead to conditions which do not facilitate microalgae growth. Specifically, the heated medium may slow microalgae growth or even stop microalgae growth altogether. Further, the excess heat may cause the medium to evaporate too quickly which would require continuous pumping of fresh medium to keep the conduits functioning properly.

Also, the scarcity of fresh water is a concern in most locations that have suitable amounts of sunlight for running an effective algae growing enterprise. Therefore, the ability to provide a source for distilled fresh water without requiring a complicated or expensive system would be welcome.

In light of the above, it is an object of the present invention to provide a system for moderating the temperature of a medium for growing microalgae. Another object of the present invention is to provide a system for distilling fresh water. Another object of the present invention is to provide a system for removing a vapor medium from a medium in a conduit for growing microalgae. Another object of the present invention is to provide a system for growing algae that utilizes a semipermeable liner to trap and distill a vapor medium. Yet another object of the present invention is to provide a system and method for moderating the temperature of a medium for growing algae that is simple to implement, easy to use, and comparatively cost effective.

### SUMMARY OF THE INVENTION

In accordance with the present invention, a system and method are provided to moderate the temperature of a medium for growing microalgae and to distill fresh water therefrom. Importantly, the system and method provide these functions without requiring any expensive or moving parts. As with a typical system for growing microalgae, a conduit is provided and filled with a medium that can support growth of microalgae. Structurally, the conduit can be considered to be defined by a high end, a low end, and two sides that extend therebetween. Further, the conduit has a bottom. For the system of the present invention, an impermeable first liner extends across the conduit and covers the bottom of the conduit. Also, a gas permeable/liquid impermeable second liner extends across the conduit and covers the bottom of the conduit. As situated, the second liner is on top of the first liner. Also, a cooling mechanism is positioned between the first liner and the second liner along the bottom of the conduit. For purposes of the system, the cooling mechanism may be cool fresh water or a cooling channel.

When the conduit is used to grow microalgae, a medium, such as unbuffered sodium bicarbonate solution, flows through the conduit. As sunlight enters the medium, a small percentage of it is converted to chemical energy by the algae, while the rest of the solar energy may be absorbed by the medium and retained as heat. When the medium becomes overheated, the excess heat may adversely affect optimal algae growing conditions in the conduit or may cause more rapid evaporation of the medium. Consequently, these conditions may cause the system to not be economically viable. However, the present system allows vapor medium to pass through the second line and come into contact with the cooling mechanism. As the vapor medium cools it condenses into fresh water. As a result, the condensed fresh water is then held between the first liner and the second liner. Thus, usable, distilled fresh water is created and the medium in the conduit is cooled by the present system.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is an overhead view of a conduit holding a medium for growing algae in accordance with the present invention; and

FIG. 2 is a cross sectional view of the conduit taken along line 2-2 in FIG. 1.

### DESCRIPTION OF THE PREFERRED EMBODIMENTS

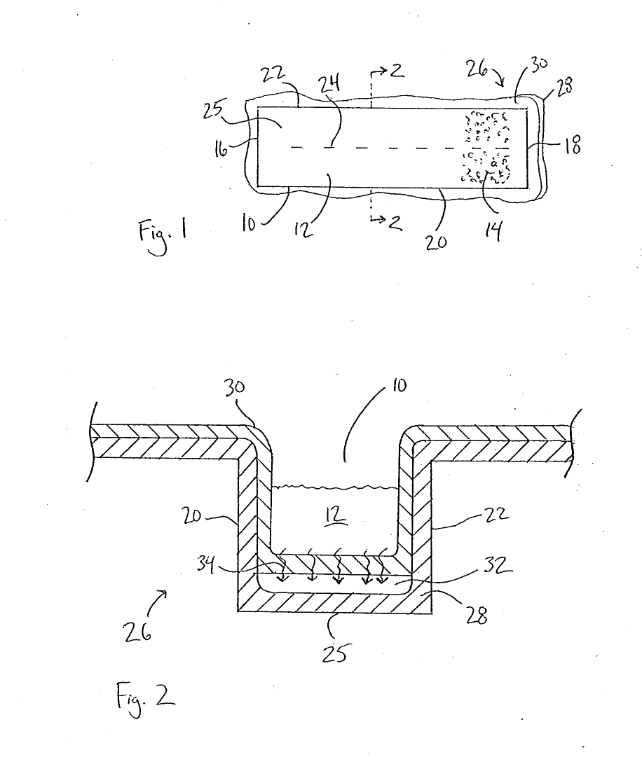

Referring to FIG. 1, a conduit for growing microalgae is shown and designated 10. As shown, the conduit 10 holds a medium 12, such as an unbuffered sodium bicarbonate solution, for supporting the growth of microalgae 14. Structurally, the conduit 10 is defined by a high end 16, from which the medium 12 flows, a low end 18, toward which the medium 12 flows, and two sides 20, 22. Further, the conduit 10 defines an axis 24 substantially parallel to the sides 20, 22, and additionally has a bottom 25.

Cross referencing FIG. 1 with FIG. 2, the system for moderating the temperature of the medium 12 is illustrated and generally designated 26. As shown, the system 26 includes an impermeable first liner 28. Structurally, the first liner 28 extends from beyond the high end 16, low end 18, and sides 20, 22 and covers the bottom 25 of the conduit 10. Further, the system 26 includes a gas permeable/liquid impermeable second liner 30 that extends from beyond the high end 16, low end 18, and sides 20, 22 and covers the bottom 25 of the conduit 10. As shown in FIG. 2, the second liner 30 is positioned on top of the first liner 28. Additionally, the system 26 provides a cooling passageway 32 between the first liner 28 and the second liner 30. Functionally, the cooling passageway 32 is intended to condense any vapor medium 12 that passes through the second liner 30 to trap the vapor medium 12 as fresh water and to cool the medium 12. In certain embodiments, the cooling passageway 32 is cool fresh water. Alternatively or additionally, the cooling passageway 32 may be a cooling channel.

During operation of the system 26, sunlight enters the medium 12. While a small percentage of the sunlight is utilized by the microalgae 14 in photosynthesis, the rest of the sunlight that is absorbed by the medium 12 is converted to heat. As a result, the medium 12 can become overheated, i.e., too hot to facilitate algae growth or so hot that there are excessive losses of water through evaporation. However, with the present system 26, a vapor from medium 12 that is created by the absorption of solar energy, can pass through the second liner 30 as indicated by the arrows 34. When the vapor from medium 12 passes through the gas permeable second liner 30, it is condensed by the cooling passageway 32 into fresh distilled water. At that point, the condensed vapor of medium 12 is trapped between the liners 28, 30 and the distilled fresh water can be suctioned or directed to a storage unit. Of course, the removal of vapor from the medium 12 also cools the medium 12.

While the particular System for Moderating the Temperature of a Medium for Growing Microalgae as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A system for controlling the temperature of a medium for growing microalgae comprising:
a conduit defined by a high end, a low end, and two sides extending therebetween, said conduit defining an axis substantially parallel to the sides and having a bottom, wherein the medium is received in the conduit to grow microalgae, said medium being heated by solar energy;
an impermeable first liner extending from beyond the high end, low end, and sides and covering the bottom of the conduit;
a gas permeable/liquid impermeable second liner extending from beyond the high end, low end, and sides and covering the bottom of the conduit, with said second liner being positioned on top of the first liner to create a passageway therebetween; and
a cooling means positioned in the passageway between the first liner and the second liner to condense any vapor medium that passes through the second liner to trap the vapor medium as fresh water and to cool the medium.

2. A system as recited in claim 1 wherein the cooling means is cool water.

3. A system as recited in claim 1 wherein the cooling means is a cooling channel.

4. A system as recited in claim 1 wherein the cooling means is cool water and a cooling channel.

5. A system as recited in claim 1 wherein the conduit is a raceway.

6. A system as recited in claim 1 wherein the conduit is a plug flow reactor.

7. A system as recited in claim 1 wherein the medium is an unbuffered solution of sodium bicarbonate.

8. A system for moderating the temperature of a medium for growing microalgae and for distilling fresh water comprising:
a conduit having a bottom, with said medium flowing through the conduit to grow microalgae while absorbing solar energy;
an impermeable first liner extending across the conduit and covering the bottom of the conduit;
a gas permeable/liquid impermeable second liner extending across the conduit and covering the bottom of the conduit, with said second liner being positioned on top of the first liner to establish a passageway therebetween; and
a means for condensing any vapor medium that passes through the second liner and into the passageway to distill the vapor medium as fresh water while cooling the medium.

9. A system as recited in claim 8 wherein the condensing means is cool water.

10. A system as recited in claim 8 wherein the condensing means is a cooling channel.

11. A system as recited in claim 8 wherein the condensing means is cool water and a cooling channel.

12. A system as recited in claim 8 wherein the conduit is a raceway.

13. A system as recited in claim 8 wherein the conduit is a plug flow reactor.

14. A system as recited in claim 8 wherein the medium is an unbuffered solution of sodium bicarbonate.

15. A method for moderating the temperature of a medium for growing microalgae and for distilling fresh water comprising the steps of:
providing a conduit having a bottom;
extending an impermeable first liner across the conduit to cover the bottom of the conduit;
extending a gas permeable/liquid impermeable second liner across the conduit and to cover the bottom of the conduit, with said second liner being positioned on top of the first liner to establish a passageway therebetween;
flowing said medium through the conduit to grow microalgae while absorbing solar energy; and
condensing any vapor medium that passes through the second liner and into the passageway to distill the vapor medium as fresh water while cooling the medium.

16. A method as recited in claim 15 further comprising the step of flowing cool fresh water between the first liner and the second liner to condense any vapor medium that passes through the second liner.

17. A method as recited in claim 15 further comprising the step of positioning a cooling channel between the first liner and the second liner to condense any vapor medium that passes through the second liner.

18. A method as recited in claim 15 wherein the conduit is a raceway.

19. A method as recited in claim 15 wherein the conduit is a plug flow reactor.

20. A method as recited in claim 15 wherein the medium is an unbuffered solution of sodium bicarbonate.
